(19) United States
(12) Patent Application Publication
(10) Pub. No.: US 2008/0195054 A1
(43) Pub. Date: Aug. 14, 2008

(54) Safety cannula needle protector

(75) Inventors: Fraser R. Snarp, Vancouver (CA); James T. Hourigan, Alpharetta, GA (US)

Correspondence Address: NIXON & VANDERHYE, PC, 901 NORTH GLEBE ROAD, 11TH FLOOR, ARLINGTON, VA, 22203, US

(73) Assignee: Inviro Medical, Inc., Lawrenceville, GA (US)

(21) Appl. No.: 12/068,601

(22) Filed: Feb. 8, 2008

Related U.S. Application Data
(60) Provisional application No. 60/900,115, filed on Feb. 8, 2007.

(51) Int. Cl. A61M 5/32

(52) U.S. Cl. 604/192

(57) ABSTRACT

A safety aspiration cannula for protecting a sharp injection needle including: a hub or base portion attachable to a syringe or injection needle hub that has an injection needle with a sharp hollow tip; a cannula body extending away from the hub or base portion and adapted to enclose the injection needle, the cannula having a tip adapted to receive a fluid and transfer the fluid to the syringe via the hollow tip of the injection needle.

This application claims priority from U.S. Provisional Application Ser. No. 60/900,115, filed Feb. 8, 2007.

### BACKGROUND OF THE INVENTION

The use of syringes with sharp metal needles carries the risk of accidental needlestick injury before, during and after administration of an injection. Rightly, much attention has been paid to reducing or eliminating accidental needlestick injuries from contaminated needles i.e., after the injection has been given and the needle has been in contact with the blood and body fluids of the patient.

Nonetheless needlestick injuries can occur prior to an injection being administered. After such an accidental needlestick, it may be necessary to discard the needle and syringe to ensure there is no transmission of blood-borne pathogens from the healthcare worker to the patient. If the syringe has already been filled with medication, this may require discarding the contents of the syringe which could be an expensive vaccine or medication.

There is a widely held perception, justified or not, that during the process of filling a syringe, the needle used to penetrate the vial stopper may become blunted, bent or in some other way altered to make subsequent penetration of the patient's skin more painful. There is no doubt that if the tip of a fine metal needle is accidentally pushed against the metal ring which surrounds and holds the vial stopper in place, it may be blunted. There is also a belief held by some healthcare workers that penetration of the vial stopper may result in the production of a small core of the stopper material which could enter the needle and presumably thereafter be injected into the patient.

There are additional concerns as well. For example, during the process of vaccination, compounds called antigens are injected into the subject with the intent of eliciting an immunological response from the subject and the production of antibodies. To assist the production of antibodies, a substance called adjuvant is often added to additionally stimulate the subject's response. If any potentially antigenic substance is injected at the same time, it is quite likely that the subject will develop an immunological or “allergic” response to that substance. At one time, the vial stoppers were made of rubber or contained latex. There has been some suggestion in medical literature that the current well-recognized and serious problem of allergy to latex may in some degree be related to the inadvertent injection of latex particles at the time of immunization, and that these particles may have come from coring of the vial stopper. This theory has gained some further credibility in the last year by the publication of at least one article describing skin sensitivity in some subjects who have been vaccinated with vaccine which had been stored in containers with butyl compound stoppers. The skin sensitivity was to the same butyl compound.

In U.S. Pat. No. 6,394,979, there is described a safety cannula with features which allow penetration of an unsupported elastomeric membrane (e.g., a vial stopper or commonly available split septum access port) but which will not easily penetrate skin or a supported elastomeric membrane (e.g., a protective elastomeric or latex glove worn on a hand). This safety cannula, once it has been used for the filling of a syringe, is then used to transfer fluid to another vial or alternatively is removed from the syringe in order that the syringe can be used in conjunction with a regular metal needle for a percutaneous injection. After removal of the cannula, the Luer lock connector of the syringe could also be used to interface with any Luer fitting mechanism such as a Luer activated valve in an intravenous infusion set. A similar device may be utilized in connection with this invention.

### BRIEF DESCRIPTION OF THE EXEMPLARY EMBODIMENT

This invention serves to protect a sharp metal needle during the process of penetration of a vial stopper and during aspiration of medication from a vial. The protection prevents damage to the needle itself and also prevents accidental needlestick injury to the healthcare worker, the patient or the family.

This is accomplished by covering or surrounding the sharp (metal, plastic or other) injection needle with a less-sharp, hollow-tipped safety cannula (sometimes referred to herein as a “protective cover” or “aspiration cannula”) with some of the properties noted above in the above-referenced US patent. As the length of the protective safety aspiration cannula is slightly greater than the length of the shaft of the injection needle, the aspiration cannula prevents any accidental contact of the sharp point of the inner injection needle with the metal ring often used to hold the vial stopper in place.

The protective cover or aspiration cannula also prevents the wiping action of the vial stopper on the injection needle which occurs during the penetration and passage of the injection needle through the vial stopper. This wiping action tends to remove or diminish the silicon applied to the needle during manufacture to facilitate passage of the needle through the patient's skin. This can result in increased drag as the needle passes through the patients tissue, making the injection more painful.

In addition, as the needle shaft and point are contained within the external safety cannula, it will prevent, probably absolutely, accidental needlestick injuries during the process of filling the syringe. If the end user is accidentally or physically jostled, or has their concentration disrupted during filling of the syringe, the needle of the syringe being filled can be accidentally dislodged from the vial. The safety cannula in this situation would protect the needle from causing a needlestick injury.

In another exemplary embodiment, a semi-sharp, solid-tip safety aspiration cannula is employed as the protective cover for the needle. The solid-tip cannula eliminates the coring problem that could occur with the first described, relatively sharper and hollow-tipped safety cannula or protective cover. By covering the sharp injection needle with a safety or aspiration cannula which has a solid tip and one or more side openings, the possibility of a production of small particles or cores of the vial stopper is almost completely eliminated.

Therefore if a solid-tip safety cannula covers the sharp injection needle during aspiration of a vaccine from a vial, and if it completely prevents contact between the sharp injection needle and a vial stopper, it is not possible that any particulates will be generated by the sharp tip of the injection needle as it penetrates the vial stopper. Further, any microscopic particles whether related to puncture of the vial stopper or not, which might otherwise have adhered to the sharp injection needle, will adhere to the safety cannula instead and will be discarded prior to injection.

Accordingly, in its broader aspects, the invention relates to a safety cannula for protecting an injection needle comprising: a hub or base portion attachable to a syringe that has an injection needle with a sharp hollow tip; a cannula body extending away from the hub or base portion and adapted to enclose the injection needle, the cannula having a tip adapted to receive a fluid and transfer the fluid to the syringe via the hollow tip of the injection needle.

In another aspect, the invention relates to a safety cannula for protecting an injection needle comprising: a hub or base portion attachable to a syringe that has an injection needle with a hollow tip; a cannula body extending away from the hub or base portion and adapted to enclose the injection needle, the cannula having a solid tip with one or more side openings therein, adapted to receive a fluid and transfer the fluid to the syringe via the hollow tip of the enclosed injection needle.

In yet another aspect, the invention relate to a syringe and safety cannula assembly comprising a syringe having a sharp, hollow-tipped injection needle; a safety cannula attached to the syringe, the safety cannula comprising a hub or base portion, a cannula body extending from the hub or base portion in surrounding relationship to the injection needle, the cannula body terminating at a tip located axially beyond the injection needle.

### BRIEF DESCRIPTION OF THE DRAWINGS

FIG. 1 is a side elevation of a safety cannula for protecting an injection needle in accordance with one exemplary, non-limiting example;

FIG. 2 is a cross-section taken along the line 2-2 of FIG. 1;

FIG. 3 is a side elevational, partly in section, illustrating the safety cannula of FIGS. 1 and 2 applied over an interchangeable needle syringe;

FIG. 4 is a side elevation, partly in section, of a safety cannula of FIGS. 1 and 2 applied over a syringe with an integral needle; and

FIG. 5 is a side elevation, partly in section, if a solid-tip safety cannula needle protector applied over a syringe and needle.

### DETAILED DESCRIPTION OF THE EXEMPLARY EMBODIMENT

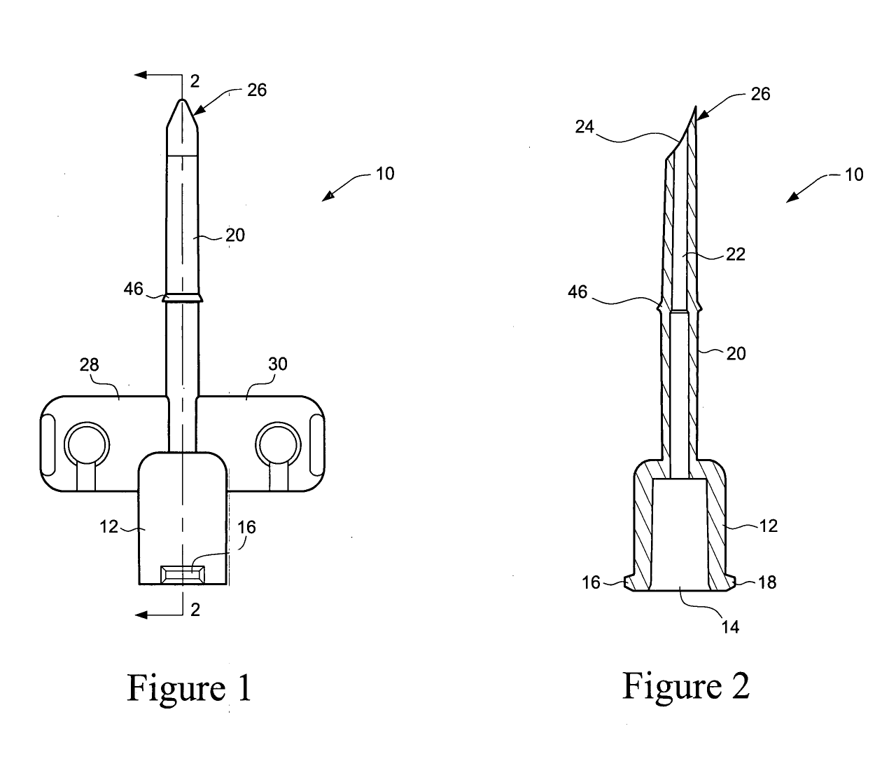

With reference initially to FIGS. 1 and 2, a safety aspiration cannula 10 for protecting a sharp, hollow-tipped metal, plastic or other injection needle 72 includes a substantially cylindrical base or hub portion 12 optionally provided with a pair of a radially outwardly extending, diametrically opposed fingers or tabs 16, 18 adjacent a first open end 14. The needle-like body 20 of the cannula is also substantially cylindrical, but with a smaller diameter than the base portion 12. The cannula is formed with an internal bore 22 that extends to a second and opposite end 24, terminating at a relatively sharply-pointed hollow tip 26 which will allow penetration of a vial stopper.

A pair of diametrically opposed wings 28, 30 are provided at the juncture of the base 12 and the cannula body 20 and provide convenient means by which the safety cannula can be attached to and removed from a syringe injection needle, as will be explained in greater detail below. These wings while they may enhance function in some embodiments, may be absent in others.

It will be appreciated that the semi-sharp tip 26 of the safety aspiration cannula allows penetration of an unsupported elastomeric membrane (for example, a vial stopper), but does not allow penetration of the skin or a supported elastomeric membrane (for example, a protective elastomeric or latex glove worn by the hand).

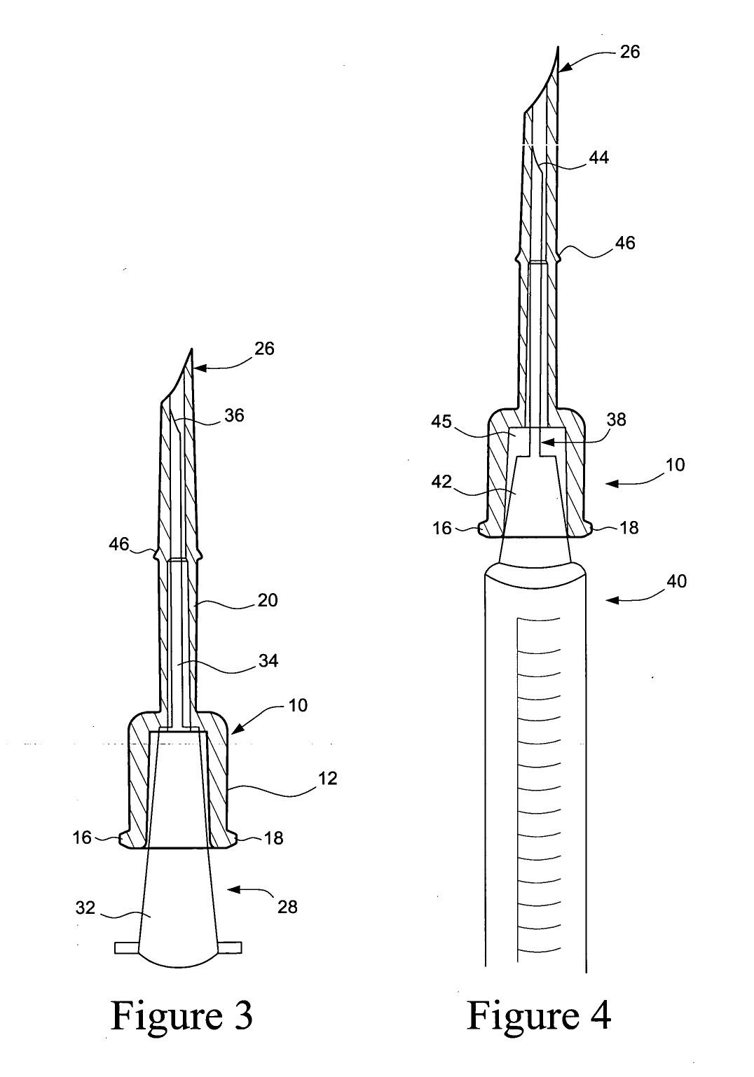

FIG. 3 illustrates the safety aspiration cannula of FIGS. 1 and 2 applied over an interchangeable or retractable syringe injection needle 28 formed with a tapered hub 32 and a needle body 34 terminating in a sharp hollow tip 36. Note that the safety cannula 10 has a length sufficient to extend beyond the tip 36 of the syringe needle 28 so as to prevent any accidental contact of the sharp point of the injection needle with, for example, a ring (typically metal) holding the vial stopper in place, or skin. As also explained above, the safety aspiration cannula prevents the wiping action which may occur during penetration and movement of the injection needle through the vial stopper. Since the needle shaft and sharp skin-penetrating point are contained within the external safety aspiration cannula, accidental needlestick injuries during the process of filling the syringe will also be prevented.

FIG. 4 illustrates the safety aspiration cannula 10 applied over an injection needle 38 that is integrally non detachably formed with a syringe 40. As in the case of the interchangeable needle (attached by a standard Luer fitting or the like), the injection needle 38 is formed with a tapered hub 42 and the needle body portion extends from the hub 42 terminating at a sharp hollow tip 44.

During the process of filling a syringe, a partial vacuum is developed in the syringe by withdrawing the syringe plunger (not shown) within the syringe barrel in a rearward direction. As there is an air and water tight seal between the injection needle hub and the syringe (usually using a Luer-type male cone and female receptacle-type connection as in FIG. 3 or by reason of an integral construction shown in FIG. 4), and as the injection needle itself is firmly sealed within the needle hub, this partial vacuum results in fluid being aspirated into the barrel of the syringe through the injection needle. For this syringe-filling procedure, the described safety aspiration cannula 10 which surrounds the injection needle 38 must have an air and water seal at the interface of the safety aspiration cannula and the injection needle hub or with the front of the body of the syringe directly.

In addition, for most circumstances, it is desirable that a small volume as possible of the aspirated fluid remain in the space 45 between the safety aspiration cannula and the injection needle and its hub after the desired volume of medication is aspirated into the syringe barrel. Therefore, in addition to a good seal between the aspiration cannula and the hub or front of the syringe barrel, it is desirable that the safety aspiration cannula conform to the external dimensions and configuration of the injection needle and its hub as closely as possible. This will tend to reduce the (dead) space 45 outside the needle and syringe to as small a volume as possible and conserve the medication or aspirated fluid.

The injection into a patient requires separation of the outer safety aspiration cannula 10 from the inner injection needle (28 or 38). In some circumstances if the full contents of a syringe have been aspirated into the syringe it may be convenient that the safety aspiration cannula remain through the stopper, attached to and discarded with the empty vial. In that case, a retention ridge 46 (FIGS. 3 and 4, seen on the outside of the shaft of the safety aspiration cannula between hub 42 and tip 44) may serve to retain the safety aspiration cannula in situ in the vial and by so doing enhance the ease of separation of the safety cannula and the injection needle (Other structural features may act in the same way as the described retention ridge).

Ridge 46 (or some similar index) on the safety aspiration cannula can also be used to assist the healthcare worker to identify the optimal penetration and positioning of the safety aspiration cannula through the vial stopper to ensure that if desired all of the contents of the vial can be removed when the vial is inverted as is customarily done to allow syringe filling.

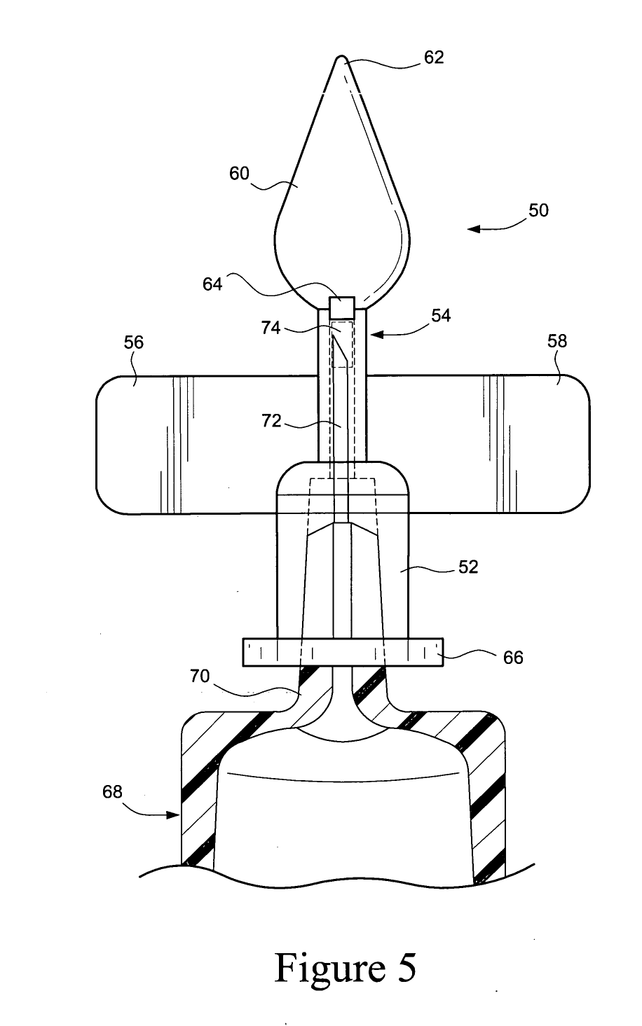

FIG. 5 illustrates another and preferred but non-limiting embodiment of a safety aspiration cannula or needle protector 50 that falls within the scope of this invention. The aspiration cannula 50 includes a substantially cylindrical hub or 52 from which extends a narrower needle-like body 54. An optional pair of radially outwardly extending wings or tabs 56, 58 are joined to the body portion. A solid tip 60 of rounded conical shape is formed at the distal end of the body 54, and is formed with a solid, semi-sharp tip portion 62, with at least one side opening 64 (two or more could also be employed) located at the juncture of the tip 60 and the body 54. At the opposite or proximate end of the protector, there is provided a radial flange 66 (either continuous or in the form of spaced circumferential tabs, similar to tabs 16, 18) at the base of the hub 52.

The safety aspiration cannula or needle protector 50 is shown installed over the tip of a syringe 68 and specifically over a narrow tip portion 70 and the injection needle 72.

In use, the solid-tip aspiration cannula 50 is used to penetrate, for example, a vial membrane, stopper or split septum access port, and fluid may be drawn through the one or more side openings 64 and then into the syringe via the opening in the tip of the syringe needle 72. The optionally enlarged head of the aspiration cannula tip 60 also serves to help to prevent accidental removal of the cannula from a vial or the like after penetration, thus eliminating the need for a separate retention device such as the ridge 46 in the embodiment shown in FIGS. 1-4.

Utilizing the solid-tip safety cannula 50 with its one or more side openings 64, it is possible to place a small hypoallergenic or other suitable plug 74, preferably of a microporous, possibly foam-like material, inside the safety cannula. Thus, when the injection needle 72 is inserted into the safety cannula 50, the tip of the sharp injection needle 72 will be surrounded by the previously inserted filtration plug. The plug may be made in the form of a cylinder of larger diameter than the needle. Alternatively, the tip of the sharp injection needle may be resting within the foam-like plug, within the safety aspiration cannula. The plug can surround the needle and extend axially along the needle as far as desired, so long as the fluid must pass through the filter to reach the tip of the syringe injection needle. In any case, the effect of this material would be to act as a filter, filtering out particles and preventing them from entering the syringe through the hollow tip of the syringe needle. At least a substantial reduction in such particulates is possible with the above arrangement.

Thus, the safety aspiration cannula may be considered to act as a filter in that most if not all particulates that do find their way into the fluid drawn from the vial, can be prevented from entering the syringe by reason of the filtering characteristics of the plug 74 or other material surrounding the syringe needle tip. The use of a filtering plug also reduces the amount of deadspace between the syringe needle and the inner surface of the safety cannula. It will be appreciated that the use of a filter plug is equally applicable to the first-described embodiment.

For both described embodiments, it will be understood that the base or hub portions of the protective safety cannulas can interface with any one or a combination of the syringe needle, the syringe hub or the forward end of the syringe barrel in any of numerous conventional techniques, and may include a standard Luer fitting or the like. It is also possible that the narrow fluid channel or lumen of the safety cannula may project farther into the solid tip 60 to accommodate longer syringe needles, and of course, the plug 74 could also be located within the extended lumen.

It can be appreciated that many different configurations of protective safety cannulae are possible within the general description above, and that the safety cannulas as described could be affixable to the syringe/needle combination or to an injection needle alone by the end user.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A safety cannula for protecting an injection needle comprising:
a hub or base portion attachable to a syringe that has an injection needle with a sharp hollow tip;
a cannula body extending away from the hub or base portion and adapted to enclose the injection needle, said cannula having a tip adapted to receive a fluid and transfer the fluid to the syringe via the hollow tip of the injection needle.

2. The safety cannula of claim 1 wherein said tip of said cannula has a semi-sharp hollow point.

3. The safety cannula of claim 1 wherein said tip of said cannula has a solid relatively sharp tip with one or more side openings therein.

4. The safety cannula of claim 1 wherein said safety cannula is provided with one or more radially extending tabs.

5. The safety cannula of claim 3 wherein said solid relatively sharp tip is enlarged relative to said cannula body.

6. The safety cannula of claim 1 including a filter located in said cannula body, adapted to enclose the hollow tip of the injection needle.

7. A safety cannula for protecting an injection needle comprising:
a hub or base portion attachable to a syringe that has an injection needle with a hollow tip;
a cannula body extending away from the hub or base portion and adapted to enclose the injection needle, said cannula having a solid tip with one or more side openings therein, adapted to receive a fluid and transfer the fluid to the syringe via the hollow tip of the enclosed injection needle.

8. The safety cannula of claim 7 wherein said safety cannula is provided with one or more radially extending tabs.

9. The safety cannula of claim 7 wherein said solid tip is enlarged relative to said cannula body.

10. A syringe and safety cannula assembly comprising a syringe having a sharp, hollow-tipped injection needle;
a safety cannula attached to said syringe, said safety cannula comprising a hub or base portion, a cannula body extending from said hub or base portion in surrounding relationship to said injection needle, said cannula body terminating at a tip located axially beyond the injection needle.

11. The assembly of claim 10 wherein said tip comprises a hollow-tip that is less sharp than said hollow-tipped injection needle.

12. The assembly of claim 10 wherein said tip comprises an enlarged, solid and semi-sharp tip with one or more side openings.

13. The assembly of claim 10 wherein said safety cannula is provided with a filter enclosing a forward end of the injection needle.

14. The assembly of claim 13 wherein said filter comprises a plug of microporous foam material.

15. The assembly of claim 10 wherein said safety cannula is attached to the syringe by a standard Luer fitting.
